(19) United States
(12) Patent Application Publication
(10) Pub. No.: US 2007/0225659 A1
(43) Pub. Date: Sep. 27, 2007

(54) Introducer sheath having frangible tip

(75) Inventor: Jeffry S. Melsheimer, Springville, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 11/725,391

(22) Filed: Mar. 19, 2007

Related U.S. Application Data
(60) Provisional application No. 60/784,171, filed on Mar. 21, 2006.

(51) Int. Cl. A61M 25/00; A61M 5/00

(52) U.S. Cl. 604/264

(57) ABSTRACT

An introducer assembly for use in delivering an implantable device to a target site within the vasculature of a patient includes an outer sheath and an inner catheter sized for passage through the lumen of the sheath. The outer sheath has a shaft portion and a tapered distal tip portion, and the inner catheter is sized for carrying the implantable device thereon. The tapered distal tip portion includes a frangible portion capable of splitting when the portion of the inner catheter having the implantable device thereon passes through the sheath distal tip portion for deployment of the implantable device at the target site.

### RELATED APPLICATION

The present patent document claims the benefit of the filing date under 35 U.S.C. §119(e) of Provisional U.S. Patent Application Ser. No. 60/784,171, filed Mar. 21, 2006, which is hereby incorporated by reference.

### BACKGROUND

1. Technical Field

This invention relates to a medical introducer apparatus. More particularly, the invention relates to an introducer sheath having a frangible distal tip portion for allowing passage therethrough of a medical device for deployment to a target site within the vasculature of a patient.

2. Background Information

Medical introducer apparatuses, such as guide catheters and introducer sheaths, are widely used in the medical field as conduits for percutaneously transporting a medical device through the vasculature of a patient to a target site for deployment. One example of a device that is frequently deployed through such apparatuses is a stent. Typically, stents are delivered through a sheath in a compressed state to a target site. Following deployment, the stent thereafter expands to substantially take the diameter of the vessel into which it is deployed. Stents delivered in this manner are typically of two general types. One type, a so-called balloon expandable stent, is delivered to the site over the balloon portion of a balloon catheter. Upon inflation of the balloon, the overlying stent expands to the outer diameter of the inflated balloon. The balloon is then deflated and retracted, leaving the stent in place at the site of deployment. The other type, a so-called self-expanding stent, is formed of a self- expandable material, such as nitinol. The stent is delivered to the site of deployment in the vessel, whereupon the stent self-expands to the desired diameter.

Often, the introducer apparatus having the stent compressed therein must traverse tortuous passageways in the patient's vasculature to reach the desired deployment site. Many designs of sheaths have been developed in an attempt to optimize this process. For example, some sheaths are formed to have different hardness levels, or durometers, along the length of the sheath. Such sheaths generally have a high durometer at the proximal end, one or more intermediate sections of increasingly lower durometer, and a distal section having the lowest durometer. This arrangement enables the distal portion of the sheath to more easily bend while traversing increasingly narrow and tortuous passageways, while maintaining a higher degree of strength and rigidity at the proximal end. Other sheaths are formed with a layered structure, which structure may include the presence of a reinforcing layer. One type of reinforcement is a helical coil that is disposed between inner and outer polymeric layers of the sheath. The presence of the coil enables the sheath to bend as it encounters a bending stress, and then return to its original orientation upon release of the stress. This type of reinforcement is generally effective for resisting bulging of the sheath adjacent to the self-expanding stent enclosed therein. Another type of reinforcement is a woven braid. A braided reinforcement enhances the torqueability of the sheath as it traverses the passageway. Sheaths may be formed to combine one or more features of the aforementioned designs, as well as other features that may be added for a particular purpose.

Although the aforementioned sheaths have proven to be useful for many intended applications, at other times the tortuousity of the vascular passageway has hindered, or even prevented altogether, the ability of the physician to direct the sheath to the target site. This difficulty is aggravated when a temporary dilator is provided inside the sheath to “fill-out” the large distal opening while the sheath is traveling to the target site. The presence of the dilator makes the sheath/dilator combination stiffer than the proposed sheath alone. On other occasions, attempts to traverse the tortuous vessels with existing sheaths have resulted in the vessel and surrounding tissue being exposed to an excessive amount of trauma. It is desired to provide a sheath for delivering a medical device to a target site within the vasculature that is capable of successfully navigating the vascular passageway, and that may be directed to the target site with a minimum of trauma to the affected tissue.

### BRIEF SUMMARY

The present invention addresses the shortcomings of the prior art. In one form thereof, the invention comprises a sheath for use in delivering an implantable device to a target area in the vasculature of a patient. The sheath comprises an elongated shaft portion having a proximal end and a distal end, and a tip portion. The tip portion has a large diameter proximal end and tapers to a small diameter distal end. The tip portion proximal end is engaged with the shaft portion distal end. The tip portion includes at least one longitudinally weakened portion extending in the proximal direction from the tip portion distal end, which tip portion is capable of splitting at the longitudinally weakened portion upon delivery of the implantable device therethrough.

In another form thereof, the invention comprises an introducer assembly for delivering an implantable device to a target site in the vasculature of a patient. The introducer assembly comprises an outer sheath having an elongated shaft portion and a tip portion. The elongated shaft portion has a proximal end and a distal end. The tip portion has a larger diameter proximal end and tapers to a smaller diameter distal end, wherein the tip portion proximal end is engaged with the shaft portion distal end. The shaft portion and the tip portion define a lumen extending axially through the sheath. An inner catheter is positioned in the sheath lumen and is capable of axial movement substantially through the lumen. The inner catheter is sized for carrying the implantable device thereon, wherein the inner catheter is structured and arranged such that upon distal movement of at least a portion of the inner catheter relative to the sheath tip portion, the tip portion distal end splits to enable passage of the inner catheter portion therethrough.

In yet another form thereof, the invention comprises a method for delivering an implantable device to a target site within the vasculature of a patient. A sheath assembly is provided for insertion into a vessel of a patient, which sheath assembly comprises a sheath and an inner catheter. The sheath includes an elongated shaft portion and a tip portion. The elongated shaft portion has a proximal end and a distal end. The tip portion has a larger diameter proximal end and tapers to a smaller diameter distal end, and the tip portion proximal end is engaged with the shaft portion distal end. The tip portion includes at least one weakened portion extending in the proximal direction from the tip portion distal end. The shaft portion and the tip portion define a lumen extending axially through the sheath, whereupon the inner catheter is disposed in the sheath lumen and is capable of axial movement therethrough. The inner catheter is sized for carrying the implantable device thereon. A portion of the inner catheter has a profile when loaded with the implantable device such that upon movement of at least the profiled portion of the inner catheter through the tip portion distal end for delivery of the device to the target site, the tip portion splits along the weakened portion. The sheath assembly is inserted into the vessel, and directed to the target site. The inner catheter is pushed in a distal direction toward the tip portion distal end such that the inner catheter profiled portion splits the tip portion along the weakened portion to enable passage therethrough of the profiled portion loaded with the implantable device. The implantable device is then deployed into the vessel.

### BRIEF DESCRIPTION OF THE DRAWINGS

FIG. 1 is a side view of one form of a sheath having a frangible tip according to the present invention;

FIG. 2 is an end view of the distal end of the sheath of FIG. 1;

FIG. 3 is a longitudinal sectional view of one embodiment of a sheath and inner catheter according to the present invention;

FIG. 4 is a longitudinal sectional view similar to the view of FIG. 3, but including an inner catheter that protrudes distally beyond the distal end of the sheath;

FIG. 5 is a longitudinal sectional view of another embodiment of a sheath and inner catheter of the present invention;

FIG. 6 is a longitudinal sectional view similar to the view of FIG. 5, wherein the inner catheter protrudes distally beyond the distal end of the sheath;

FIG. 7 is a longitudinal sectional view of still another embodiment of the sheath and inner catheter of the present invention;

FIG. 8 is a longitudinal sectional view similar to the sheath of FIG. 7, wherein the inner catheter protrudes distally beyond the distal end of the sheath;

FIG. 9 is a perspective view of the distal end portion of a frangible sheath according to the present invention, illustrating the passage of the inner catheter through the frangible distal tip of the sheath;

FIG. 10 is a sectional view of a mold suitable for use in forming a sheath frangible tip;

FIG. 11 is an end view of the mold of FIG. 10; and

FIGS. 12 and 13 are alternative embodiments illustrating cutting members arranged along the inner catheter.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of an introducer sheath, as well as-the axial ends of other component features of the invention. The term “proximal” is used in its conventional sense to refer to the end of the introducer sheath, or component, that is closest to the operator during use of the assembly. The term “distal” is used in its conventional sense to refer to the end of the sheath, or component, that is initially inserted into the patient, or that is closest to the patient.

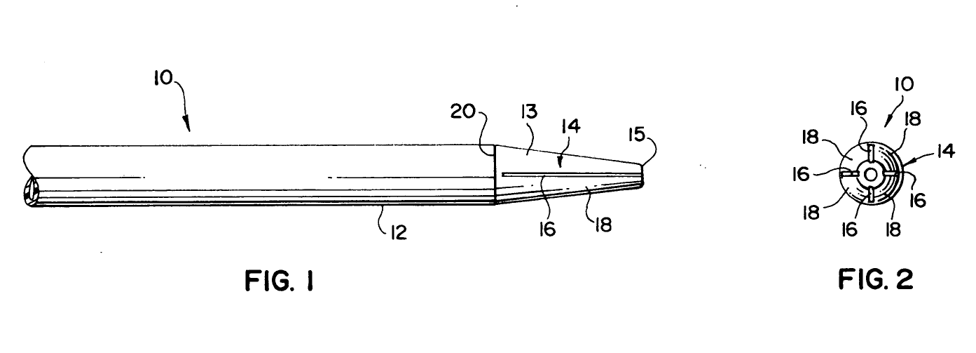

FIG. 1 illustrates a side view of a portion of an introducer sheath 10, according to an embodiment of the present invention. Sheath 10 comprises a generally cylindrical main shaft 12, and a tapered distal tip portion 14 disposed at the distal end of shaft 12. Only the distal most portion of sheath 10 is illustrated in FIG. 1. The remaining portions of the introducer sheath not illustrated are conventional, and need not be further shown and described to gain a full understanding of the features of the present invention.

Main shaft portion 12 of introducer sheath 10 may comprise any well-known guide catheter or sheath commonly used for delivering a medical device to a target site. A particularly preferred sheath for use in the present invention comprises the FLEXOR® sheath, available from Cook Incorporated, of Bloomington, Ind. The FLEXOR® sheath is a multi-layer sheath having an inner PTFE liner, an outer layer formed of a polyether block amide, such as PEBAX®, and a helical coil embedded between the layers. The outer layer may include one or more discrete PEBAX® sections that vary in durometer from a higher durometer at the proximal end of the sheath to a lower durometer at the sheath distal end. The design of the FLEXOR® sheath provides optimal flexibility with maximum resistance to kinking and compression.

Distal tip portion 14 of sheath 10 may be formed as an integral part of the sheath, or alternatively, may be separately formed and thereafter attached to the distal-most end of the shaft 12. When formed as an integral part of the sheath, a distal portion of the sheath generally free of reinforcement is typically molded to impart the desired shape. When formed separately, the distal tip portion is molded or otherwise formed to a desired size and shape, and thereafter bonded or otherwise attached (such as by gluing) to the distal-most end of shaft 12. Formation and/or attachment of the distal tip portion is further described hereinafter. As illustrated, the distal tip portion 14 tapers from a larger proximal diameter to a smaller distal diameter. It is a key feature of the present invention to utilize a sheath having a tapered distal tip. The use of a tapered tip facilitates distal movement of the sheath through the vasculature. Such a tip has the structural integrity necessary to negotiate tortuousity, and to deflect the sheath as it traverses the vasculature.

As illustrated in FIGS. 1 and 2, tapered distal tip portion 14 is provided with one or more longitudinally weakened areas 16. Longitudinally weakened areas 16 extend axially in the proximal direction from distal end 15 of the distal tip portion. Preferably, weakened areas 16 comprise respective radial slits, or score lines, that extend partially through distal tip portion 14, or in other words, from the outer surface of tip portion 14 but stopping short of the inner surface. Alternatively, the weakened areas 16 can extend all the way through distal tip 14 from the outer surface of the tip portion to the inner surface. As a still further alternative, weakened areas 16 can extend radially outward from the inner surface of tip portion 114, stopping short of the outer surface of the tip. Preferably, the weakened areas extend all, or substantially all, of the way from distal end 15 to a junction 20 of shaft 12 and distal tip portion 14.

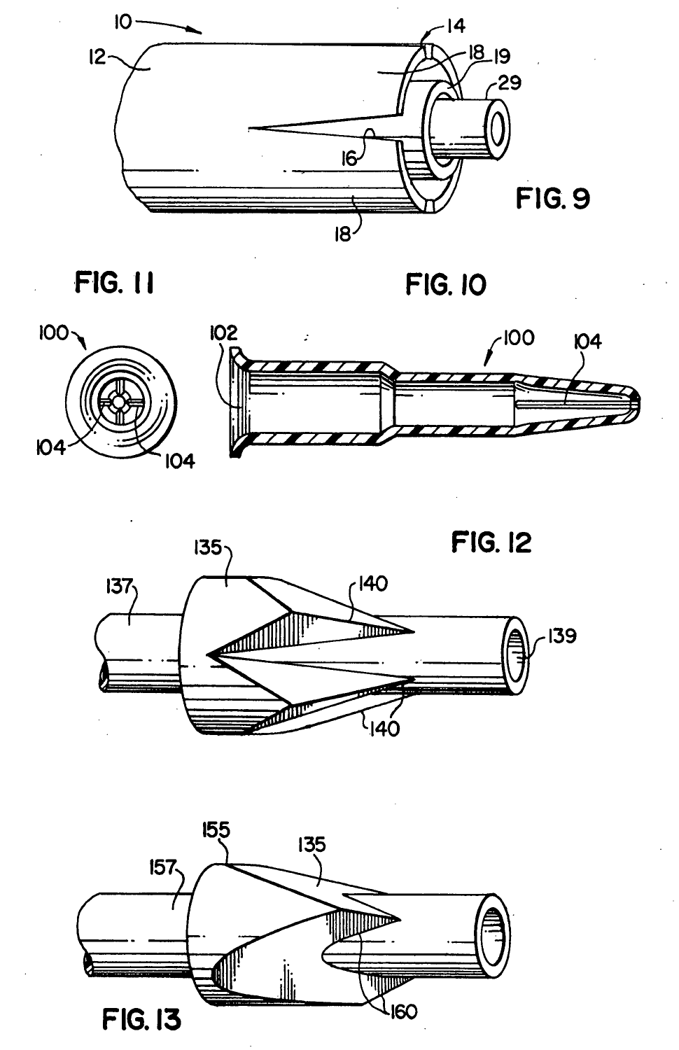

Longitudinally weakened areas 16 enable the distal tip portion to be frangible upon exposure to a stressor. Thus, upon passage of a medical device through tapered distal tip portion 14, the tip splits along the weakened areas into a plurality of discrete axial segments that extend from the distal end 15 substantially to junction 20. FIG. 9 illustrates a perspective view of a sheath having a frangible tip 14 according to an embodiment of the present invention, wherein an inner delivery catheter 29 carrying a medical device 19 for implantation is passing through tapered tip portion 14 of sheath 10.

The embodiment of sheath. 10 illustrated in the figures includes four longitudinally weakened areas 16, spaced about 90 degrees apart along the surface of the distal tip. Upon passage of catheter 29 through sheath 10, tapered distal tip portion 14 axially splits into four segments 18 (two of which are visible in the orientation of FIG. 9), each segment representing the surface area of tip portion 14 that is situated between adjacent slits, or weakened areas, 16. Those skilled in the art will appreciate that the distal tip portion need not have exactly four weakened areas, nor do the weakened areas need to be equally spaced along the surface of the tip as shown. Rather, any number or form of weakened areas 16 may be provided, and the weakened areas may be spaced in any manner desired, with the embodiment shown merely representing one possible arrangement.

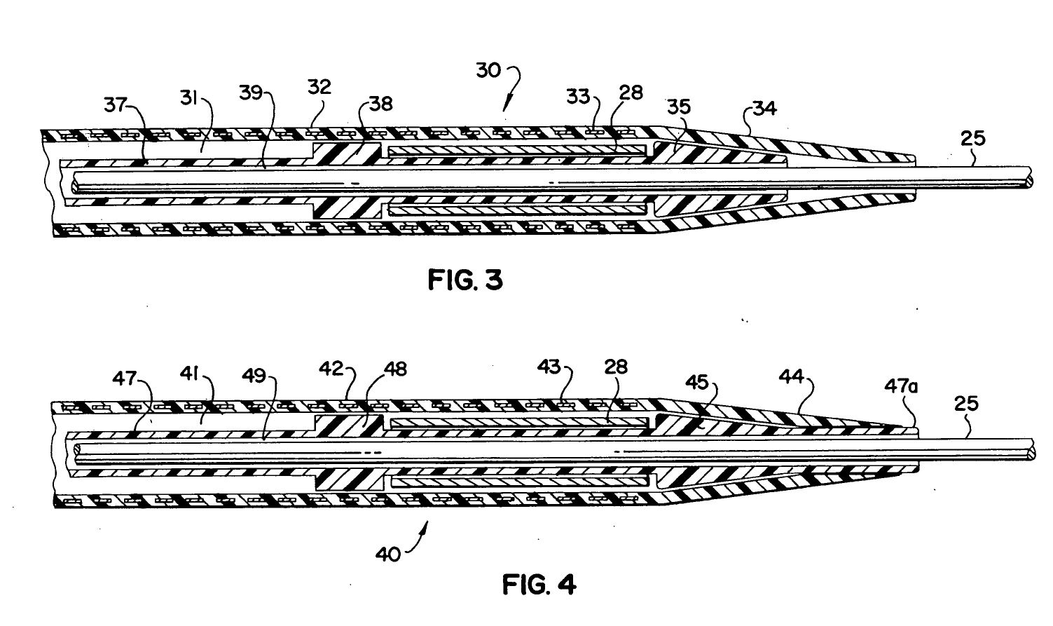

FIG. 3 illustrates a longitudinal sectional view of one embodiment of an introducer sheath 30 according to the present invention. Sheath 30 includes main shaft portion 32 and tapered distal tip portion 34. Main shaft portion 32 includes an optional reinforcing member. In this embodiment, the reinforcing member comprises a helical coil 33. Preferably, helical coil 33 is formed from a flat wire, such as stainless steel. Reinforcing members for a sheath are well known in the art, and typically comprise either a helical coil or a woven braid. Reinforcements are typically formed of metal or a metal alloy, such as stainless steel and the shape memory alloy nitinol, although reinforcements may also be formed from other suitable compositions, such as polymers and various composite formulations. Those skilled in the art can readily select an appropriate reinforcement for a particular use without undue experimentation.

In the embodiment of FIG. 3, the introducer sheath includes an inner catheter 37 that extends through the lumen 31 of the sheath. Inner catheter 37 can comprise a conventional delivery catheter for use in delivering an implantable medical device to a target site within the body of a patient. An implant, such as stent 28, is compressed in conventional fashion onto an outer surface of inner catheter 37 for delivery to the target site. In the embodiment shown, inner catheter 37 includes a pusher portion 38 proximal of the loaded stent, and a large diameter portion, such as conical portion 35, distal of the stent. Inner catheter 37 also includes a lumen 39 extending therethrough. Lumen 39 is sized and shaped for passage of a wire guide 25 therethrough. Those skilled in the art will appreciate that the delivery catheter shown is only one of many types of delivery catheters that may be utilized in the inventive apparatus.

FIG. 4 is a longitudinal sectional view of an embodiment of an introducer sheath 40 generally similar to the sheath of FIG. 3. Sheath 40 includes main shaft portion 42 and tapered distal tip portion 44. Main shaft portion 42 also includes an optional reinforcing member 43. Inner catheter 47 extends through the sheath lumen 41, and stent 28 is compressed onto inner catheter 47. Inner catheter 47 includes pusher portion 48 proximal of the stent, and conical portion 45 distal of the stent. Inner catheter 47 also includes a lumen 49 for passage of wire guide 25. Unlike the embodiment of FIG. 3, a distal end 47a of inner catheter 47 extends incrementally (such as 1 or 2 mm) beyond the distal end of the sheath. The exposed distal end 47a facilitates backloading of the wire guide into the sheath.

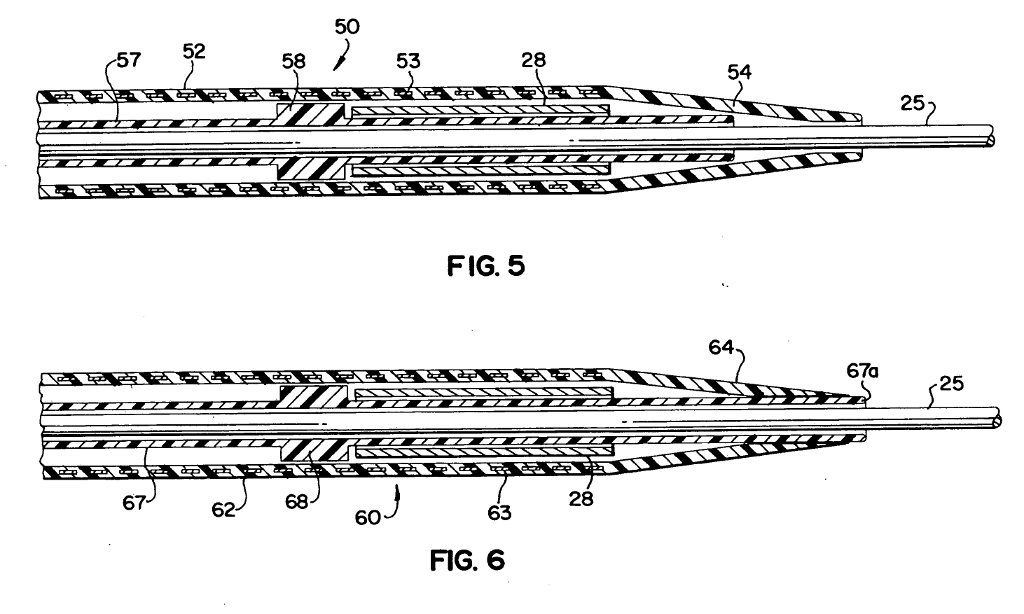

FIGS. 5 and 6 illustrate alternative embodiments of the present invention. In the embodiment of FIG. 5, sheath 50 is generally similar to sheath 30 of FIG. 3. Similarly, sheath 60 in FIG. 6 is generally similar to sheath 40 of FIG. 4. Sheaths 50, 60 include respective shaft portions 52, 62 and tapered distal tip portions 54, 64 as in the previous embodiments. Similarly, sheaths 50, 60 may include optional reinforcing members 53, 63. Inner catheters 57, 67 may include respective pusher portions 58, 68. In FIG. 6, a distal end 67a of inner catheter 67 extends incrementally beyond the distal end of sheath 60, in the same manner that distal end 47a extends beyond the distal end of sheath 40 in FIG. 4.

Unlike the embodiments of FIGS. 3 and 4, however, inner catheters 57, 67 do not include a conically-shaped distal end portion in the nature of conical portions 35, 45 of FIGS. 3 and 4, respectively. By eliminating this feature, inner catheters 57, 67 are simpler to produce than inner catheters 37, 47. In addition, with the elimination of the conical portions, introducer sheaths 50, 60 can be made shorter than corresponding sheaths 30, 40. As still another advantage, the withdrawal of the sheath during deployment of the stent is facilitated because there is no substantial structure that must be traversed or overcome as the sheath is withdrawn in the proximal direction. However, as a trade-off, with the structure shown in FIGS. 5 and 6, as stent 28 is deployed, it will bear directly against the inside surface of respective tapered portions 54, 64 as the sheath is withdrawn. Thus, in this embodiment, the stent is the structure that is primarily responsible for causing the weakened areas of the tapered tip to split. Care must be exercised to insure that the stent has the structural integrity to resist damage as a result of the contact with the inside surfaces of the tapered portions during withdrawal of the sheath.

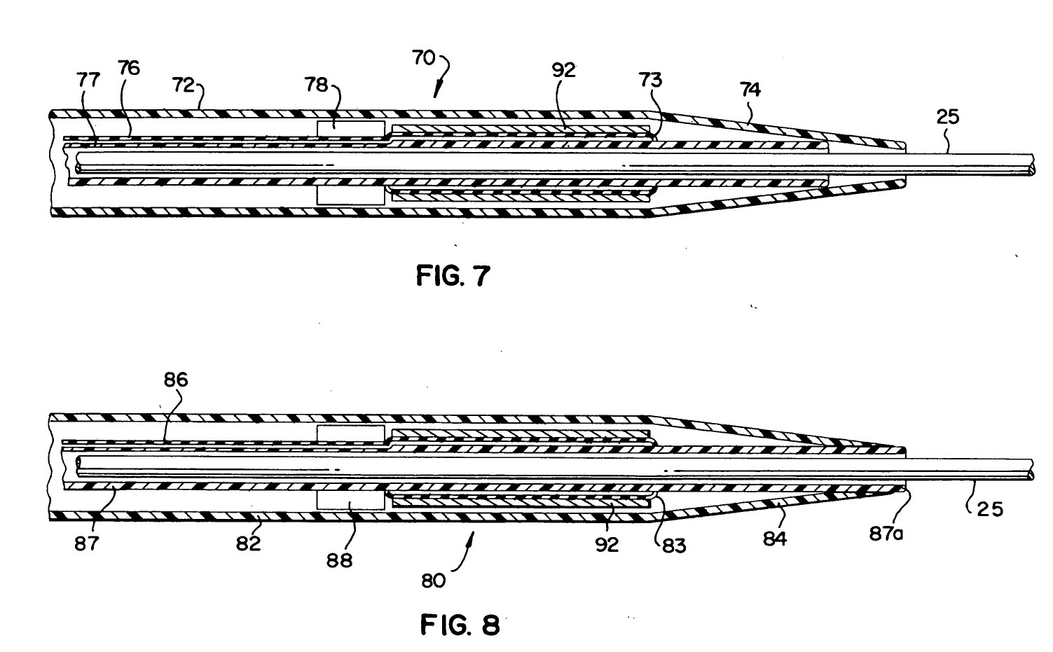

FIGS. 7 and 8 illustrate other alternative embodiments of the present invention. In these embodiments, the introducer sheath and inner catheter are structured for deployment of a balloon expandable stent 92. Sheath 70 of FIG. 7 may be generally similar to sheaths 30 and 50 in FIGS. 3 and 5, respectively. Similarly, sheath 80 of FIG. 8 may be generally similar to sheaths 40 and 60 of FIGS. 4 and 6, respectively. Sheaths 70, 80 include respective shaft portions 72, 82 and tapered distal tip portions 74, 84 as in the previous embodiments. In the embodiments shown, sheaths 70, 80 do not include the optional reinforcements. Those skilled in the art recognize that the presence or absence of a reinforcement is a routine design choice, and either alternative is considered within the scope of the present invention.

Inner balloon catheters 77, 87 may include respective pusher portions 78, 88 as before. In FIG. 8, a distal end 87a of inner balloon catheter 87 extends incrementally beyond the distal end of the sheath, in the same manner as ends 47a and 67a in the embodiments of FIGS. 4 and 6, respectively. Inner balloon catheters 77, 87 include inflatable balloons 73, 83 at a distal end thereof for use in expanding balloon-expandable stent 92 in well-known fashion. Each balloon catheter 77, 87 includes an inflation lumen 76, 86. Each lumen 76, 86 communicates at a distal end with the interior of respective balloon 73, 83, and at a proximal end with a source of inflation fluid (not shown).

The balloon catheters 77, 87 loaded with stent 92 illustrated in FIGS. 7 and 8 are over-the-wire catheters. However, the invention may likewise utilize rapid-exchange catheters instead of the over-the-wire catheters shown. Over-the-wire catheters and rapid-exchange catheters are well known in the art, and further discussion of these catheters is not required for an understanding of the present invention.

With the embodiments of FIGS. 7 and 8, sheath 70, 80 can be used alone as the initial guiding catheter having a distal opening just large enough for the wire guide to pass through. The sheath is threaded through the vasculature in the normal manner to the target site. The balloon catheter having the balloon expandable stent loaded thereon may then be inserted into the proximal opening of the sheath, and directed to the previously-reached target site. The balloon catheter with the loaded stent is then pushed distally out the distal end of the sheath, thereby causing the weakened areas to break apart, in the manner indicated in FIG. 9. Alternatively, the sheath may be pulled in the proximal direction over the balloon catheter, forcing the stent out the distal end of the sheath for deployment.

It is desirable that the frangible tip of the introducer sheath exhibit a non-injurious taper prior to deployment of the enclosed implantable device. Thus, when initiating deployment of the implantable device, the tip will split at the pre-weakened portions to allow the unimpeded passage of the device. As stated, the tapered frangible tip may be formed at the time of formation of the shaft, or alternatively, it may be formed separately, and thereafter attached to the sheath. In either event, the tip is formed to include the desired taper, as well as the weakened portions. The following discussion describes preferred methods for forming the tapered tip.

Initially, a suitable tip mold is provided. FIGS. 10 and 11 illustrate a preferred tip mold 100. Tip mold 100 consists primarily of a female mold which imparts a desired shape to the exterior of the sheath. It is desirable to also utilize a mandrel to support the interior of the sheath during the tip formation process. Preferably, the female mold is formed of an appropriate metal to facilitate resistance or convection heat-forming, or to facilitate induction heating through the application of RF energy. The cavity 102 may be shaped by use of a “plunge EDM” electrode. As another alternative, the female mold may be made of glass. Glass forming techniques are well known in the art, and a discussion of glass molds is not required for an understanding of the present invention. The glass mold can also be used for convection or resistance heat forming. The mold includes a plurality of projections 104 for use in shaping the weakened areas 16 of the sheath distal tip.

When an existing sheath distal end is to be remolded, a given sheath of “raw material” is prepared. This may include cutting the sheath to length, forming with some layers set-back from the distal end (e.g. FLEXOR®-type or other sheaths having a reinforcing layer), or special cleaning/surface preparation. Heat and/or pressure may be applied to force the introducer sheath to take on the desired shape.

For external molding of the distal tip area of an existing sheath, in the event that the implant cannot be back-loaded proximally, the tip remolding will need to take place with the implant already loaded into the introducer. Therefore, a female mold is used which will only act upon the accessible exterior of the introducer. To prevent prolapse of the introducer material, it is desirable that the inner catheter be structured to include suitable support members, such as conical portions 35, 45, shown in FIGS. 3 and 4, respectively, to support the inner diameter of the introducer during the tip remolding process. When present, the conical portions can also be used to assist in forming the tip to the desired shape.

If the implant can be back-loaded proximally, the tip remolding can take place before the implant is loaded. In this case, a female mold is used to shape the exterior of the introducer, while a shaped mandrel may be inserted inside the introducer to control and/or support the interior of the introducer during the remolding process.

When a separate, pre-formed tip is to be attached to an existing shaft, the shaft is initially prepared by cutting to length, chamfering, forming with some layers set back, or other appropriate cleaning/surface preparation. Those skilled in the art are well aware of suitable techniques for preparing surfaces for joinder to one another. The tip itself is formed of a compatible material to facilitate attachment with the shaft, which material is capable of providing the frangible performance necessary. To enhance bonding, the tip is preferably formed of the same or a similar material as the shaft. Non-limiting examples of such materials include a polyether block amide, nylon, urethanes, vinyls, and other biocompatible thermoplastics known for such medical uses. Similarly, known splittable compositions, such as PTFE, may be utilized. Splittable polymeric compositions typically comprise stress oriented polymers. These compositions are well known in the medical arts for use in splitting or otherwise peeling a device away from a substrate.

If the implant is to be pre-loaded into the introducer sheath, it may be desirable to use an adhesive to attach the pre-formed tip to the loaded sheath. Although heat bonding may also be utilized as an attachment method, the use of heat may be undesirable when the stent is loaded into the sheath at the time of formation of the tip. Preferably, the implant is retracted in the proximal direction prior to attachment of the tip to remove it from the immediate vicinity of the area to be bonded. Once the adhesive and/or heat has been applied, and the joint formed thereby has been smoothed and cured (if necessary), the implant can be re- advanced in the shaft.

If the implant can be back-loaded proximally, the pre-formed tip can be heat-bonded onto the introducer sheath. In this event, the tip and the sheath are loaded onto a mandrel in the proper relationship to one another. The assembly is thereafter advanced into a female mold. Heat and/or pressure may then be applied in well-known manner to facilitate a heat-bond between the tip and the sheath.

FIGS. 12 and 13 illustrate alternative embodiments of the invention.

FIG. 12 illustrates the distal end portion of an inner catheter 137 that includes conical portion 135. Lumen 139 extends through the inner catheter. Inner catheter 137 and conical portion 135 extend through the lumen of a sheath (not shown) in the same manner that inner catheter 37 and conical portion 35 extend through sheath 30 of FIG. 3. However, in this embodiment, conical portion 135 is configured to have a plurality of distally tapering cutters 140 along a portion of its outer surface. Cutters 140 are sized and positioned around inner catheter 137 in a manner such that the tapered distal end of the sheath (e.g., tapered end 34 of sheath 30) is weakened by the action of cutters 140 on its inner surface as the catheter is urged distally through the sheath. The weakening enables the distal tip to split in the manner shown in FIG. 9, in the same manner as the previous embodiments.

FIG. 13 is an embodiment similar to that of FIG. 12, illustrating the distal portion of inner catheter 157 that includes conical portion 155. Conical portion 155 is configured to have a plurality of tapering cutters 160 along a portion of its outer surface in the same manner as cutters 140 of FIG. 12. Cutters 140, 160 are merely two non-limiting examples of possible arrangements and orientations of cutting mechanisms that can be included on the inner catheter. Those skilled in the art will appreciate that numerous other arrangements can be substituted to achieve the same cutting effect, which alternative arrangements are considered within the scope of the invention.

When the embodiments of FIGS. 12 and 13 are utilized, it is not necessary to have a pre-scored or pre-weakened distal tip, in the manner of the previous embodiments, as such weakening is accomplished by the action of the cutters. However, if desired, the inventive introducer assembly can combine both a sheath having a weakened distal tip and an inner catheter having the cutters as described.

It is intended that the foregoing detailed description be regarded as illustrative rather than limiting, and that it be understood that it is the following claims, including all equivalents, that are intended to define the spirit and scope of this invention.

1. A sheath for delivering an implantable device to a target area in the vasculature of a patient, comprising:
an elongated shaft portion having a proximal end and a distal end; and
a tip portion, said tip portion having a larger diameter proximal end and tapering to a smaller diameter distal end, said tip portion proximal end engaged with said shaft portion distal end, said tip portion including at least one weakened portion extending in the proximal direction from said tip portion distal end, said tip portion capable of splitting at said weakened portion upon delivery of said implantable device therethrough.

2. The sheath of claim 1, wherein said tip portion includes a plurality of longitudinally weakened portions extending in the proximal direction from said tip portion distal end, wherein said tip portion is capable of splitting at said longitudinally weakened portions upon delivery of said implantable device.

3. The sheath of claim 2, wherein said weakened portions comprise radial slits extending inwardly from an outer surface of said tip portion.

4. The sheath of claim 3, wherein said radial slits terminate short of an inner surface of said tip portion.

5. The sheath of claim 2, wherein said longitudinally weakened portions extend in the proximal direction substantially to an area of engagement of said shaft portion distal end and said tip portion proximal end.

6. The sheath of claim 2, wherein said longitudinally weakened portions are substantially spaced an equal distance along a surface of said tip portion.

7. The sheath of claim 6, comprising four weakened portions spaced about 90 degrees apart along a surface of said tip portion.

8. An introducer assembly for delivering an implantable device to a target site in the vasculature of a patient, comprising:
an outer sheath, said sheath comprising an elongated shaft portion and a tip portion, said elongated shaft portion having a proximal end and a distal end, said tip portion having a larger diameter proximal end and tapering to a smaller diameter distal end, said tip portion proximal end engaged with said shaft portion distal end, said shaft portion and said tip portion defining a lumen extending axially through said sheath; and
an inner catheter disposed in said sheath lumen and capable of axial movement substantially through said lumen, said inner catheter sized for carrying said implantable device thereon, said inner catheter with said implantable device carried thereon structured and arranged such that upon distal movement of at least a portion of said inner catheter relative to said sheath tip portion said tip portion distal end splits for passage of said inner catheter portion therethrough.

9. The introducer assembly of claim 8, wherein said tip portion includes at least one weakened portion extending in the proximal direction from said tip portion distal end, said tip portion capable of splitting at said weakened portion upon passage of said inner catheter portion therethrough.

10. The introducer assembly of claim 9, wherein said inner catheter includes a generally conical portion distal of a portion of said inner catheter adapted for carrying said implantable device, said generally conical portion having a large diameter portion exceeding a diameter of said distal end of said tapered tip portion.

11. The introducer assembly of claim 9, wherein said inner catheter includes a generally conical portion distal of a portion of said inner catheter adapted for carrying said implantable device, said generally conical portion having a large diameter portion exceeding a diameter of said distal end of said tapered tip portion, said inner catheter further comprising a distal end portion extending through said distal end of said tip portion.

12. The introducer assembly of claim 8, further comprising an inflatable balloon positioned at a distal portion of said inner catheter, and an inflation lumen positioned for carrying an inflation fluid to an interior portion of said balloon for inflation thereof, said inflatable balloon oriented on said inner catheter for carrying said implantable device.

13. The introducer assembly of claim 12, wherein said inner catheter includes a generally conical portion distal of said inflatable balloon, said generally conical portion having a large diameter portion exceeding a diameter of said distal end of said tip portion.

14. The introducer assembly of claim 8, wherein said inner catheter includes a pusher portion proximal of a portion of said inner catheter carrying said implantable device.

15. The introducer assembly of claim 8, wherein said inner catheter comprises at least one cutter distal of a portion of said catheter assembly adapted for carrying said implantable device, said at least one cutter structured and arranged such that upon said relative distal movement said cutter forms a weakened portion in said tip portion distal end.

16. The introducer assembly of claim 15, wherein said inner catheter comprises a generally conical portion distal of said portion adapted for carrying said implantable device, and wherein said at least one cutter extends longitudinally along a surface of said generally conical portion.

17. A method for delivering an implantable device to a target site within the vasculature of a patient, comprising:
providing an introducer assembly for insertion into a vessel of a patient, said introducer assembly comprising a sheath and an inner catheter, said sheath comprising an elongated shaft portion and a tip portion, said elongated shaft portion having a proximal end and a distal end, said tip portion having a larger diameter proximal end and tapering to a smaller diameter distal end, said tip portion proximal end engaged with said shaft portion distal end, said tip portion including at least one longitudinally weakened portion extending in the proximal direction from said tip portion distal end, said shaft portion and said tip portion defining a lumen extending axially through said sheath; said inner catheter disposed in said sheath lumen and capable of axial movement therein, said inner catheter sized for carrying said implantable device thereon, a portion of said inner catheter having a profile when carrying said implantable device such that upon movement of at least said profiled portion of said inner catheter through said tip portion distal end for delivery of said device to said target site, said tip portion splits along said at least one weakened portion;
inserting a distal end portion of said introducer assembly into said vessel;
advancing said introducer assembly distal end portion to said target site;
moving said inner catheter in a distal direction relative to said tip portion distal end such that said inner catheter profiled portion splits said tip distal end along said at least one weakened portion to enable passage therethrough of said profiled portion carrying said implantable device; and
deploying said implantable device from said inner catheter into said vessel.

18. The method of claim 17, wherein said implantable device comprises an expandable stent, and said deploying step comprises delivery of said stent to said target site, and expanding said stent from a compressed condition to an expanded condition.

19. The method of claim 18, wherein said inner catheter comprises an inflatable balloon, and said stent is expanded by inflating said balloon.

20. The method of claim 17, wherein said inner catheter comprises a generally conical portion distal of a portion of said inner catheter adapted for carrying said implantable device, said generally conical portion having a large diameter portion exceeding a diameter of said distal end of said tapered tip portion.
